(19) United States
(12) Patent Application Publication
(10) Pub. No.: US 2024/0049785 A1
(43) Pub. Date: Feb. 15, 2024

(54) BATTERY MODULE AND ATOMIZER

(71) Applicant: Luxshare Precision Industry Company Limited, Shenzhen (CN)

(72) Inventors: Huabing LI, Shenzhen (CN); Zhongyuan LAI, Shenzhen (CN); Hongbing YIN, Shenzhen (CN); Lei HE, Shenzhen (CN); Yu HUANG, Shenzhen (CN)

(21) Appl. No.: 18/210,357

(22) Filed: Jun. 15, 2023

(30) Foreign Application Priority Data
Aug. 10, 2022 (CN) 202210955843.1

(51) Int. Cl. A24F 40/40; H01M 50/247; H01M 50/284; H02J 7/00; H01M 50/519; H01M 50/522

(52) U.S. Cl. CPC A24F 40/40; H01M 50/247; H01M 50/284; H02J 7/0044; H01M 50/519; H01M 50/522; H01M 2220/30

(57) ABSTRACT

A battery module includes a battery, a main control board, and an elastic conduction sheet. The elastic conduction sheet has elasticity. The battery is electrically connected to the main control board by the elastic conduction sheet. One end of the elastic conduction sheet is welded to the battery and another end of the elastic conduction sheet is welded to the main control board. An atomizer includes an atomization compartment and the preceding battery module. The battery module is connected to the atomization compartment and configured to supply power to the atomization compartment.

### CROSS-REFERENCE TO RELATED APPLICATION

This application claims priority to Chinese Patent Application No. 202210955843.1 filed Aug. 10, 2022, the disclosure of which is incorporated herein by reference in its entirety.

### TECHNICAL FIELD

The present disclosure relates to the technical field of atomizers, for example, a battery module and an atomizer.

### BACKGROUND

An atomizer is used for atomizing a test solution. According to different uses, an atomizer includes an air humidifier, a medical nebulizer, an electronic cigarette, or an atomization device for beauty and cosmetics.

The battery and the main control board of the power supply module in an atomizer are electrically connected by lines. The lines are connected to the battery and the main control board by tin welding. The costs of tin are relatively high, resulting in high material costs. Moreover, because the ends of the lines are thin and difficult to position, welding cannot be performed by automation. Traditional manual welding is required, resulting in low efficiency.

### SUMMARY

The present disclosure provides a battery module and an atomizer, which can reduce the material costs and can be manufactured using automation equipment, thereby increasing the productive efficiency.

An embodiment of the present disclosure provides a battery module. The battery module includes a battery, a main control board, and an elastic conduction sheet. The elastic conduction sheet has elasticity. The battery is electrically connected to the main control board by the elastic conduction sheet. One end of the elastic conduction sheet is welded to the battery and another end of the elastic conduction sheet is welded to the main control board.

In an embodiment of the present disclosure, the middle portion of the elastic conduction sheet is concave and deformed toward one side to form an elastic bypass slot. Two ends of the elastic conduction sheet are capable of being elastically deformed along the direction toward the elastic bypass slot or the direction away from the elastic bypass slot.

In an embodiment of the present disclosure, the elastic bypass slot is provided with a through slot.

In an embodiment of the present disclosure, an elastic fastening member and a spring contact terminal are included. The elastic fastening member is connected to the main control board. The tail of the spring contact terminal is elastically clamped in the elastic fastening member. The head of the spring contact terminal is configured to contact a power-to-be-supplied component.

In an embodiment of the present disclosure, the elastic fastening member includes a baseplate and two side plates. The baseplate and the two side plates are connected to form a U-shaped structure. The two side plates are connected to the main control board. The two side plates are each provided with an elastic clamping plate. The tail of the spring contact terminal is inserted in the U-shaped slot of the U-shaped structure and clamped by two elastic clamping plates of the two side plates.

In an embodiment of the present disclosure, a charging connector and an electrical connector are included. The charging connector and the main control board are disposed at two ends of the battery respectively. The charging connector is electrically connected to the main control board by the electrical connector. One end of the electrical connector elastically abuts the main control board. Another end of the electrical connector is connected to the charging connector.

In an embodiment of the present disclosure, the electrical connector includes a body and an elastic abutment member. One end of the body is connected to the charging connector. One end of the elastic abutment member is connected to another end of the body. Another end of the elastic abutment member is bent and deformed to form an elastic abutment end. The elastic abutment end elastically abuts the main control board.

In an embodiment of the present disclosure, a shockproof pad is included. The shockproof pad is disposed between the charging connector and the battery.

In an embodiment of the present disclosure, the elastic conduction sheet is made of iron.

An embodiment of the present disclosure provides an atomizer. The atomizer includes an atomization compartment and the preceding battery module. The battery module is connected to the atomization compartment and configured to supply power to the atomization compartment.

### BRIEF DESCRIPTION OF DRAWINGS

FIG. 1 is a view of a battery module according to an embodiment of the present disclosure.

FIG. 2 is an exploded view of a battery module according to an embodiment of the present disclosure.

FIG. 3 is a partial exploded view of a battery module according to an embodiment of the present disclosure.

FIG. 4 is a view of an elastic conduction sheet according to an embodiment of the present disclosure.

FIG. 5 is a partial exploded view of a battery module according to an embodiment of the present disclosure.

FIG. 6 is a view of an electrical connector according to an embodiment of the present disclosure.

FIG. 7 is a view of an elastic fastening member according to an embodiment of the present disclosure.

### REFERENCE LIST

10 housing
20 holder
30 battery cover member
40 main control board cover plate
50 light-transmissive member
201 battery accommodation slot
202 main control board accommodation slot
203 insertion slot
204 opening
205 electrical connector accommodation slot
206 integrated element accommodation slot
1 battery
2 elastic conduction sheet
3 main control board
5 charging connector
6 electrical connector
7 shockproof pad
8 elastic fastening member
9 spring contact terminal
21 elastic bypass slot
22 through slot
41 integrated element
42 soft rubber sealing pad
43 first magnet
44 indicator light
61 elastic abutment member
62 body
63 fixed portion
64 fastener
81 baseplate
82 side plate
611 elastic abutment end
621 protrusion
821 elastic clamping plate
822 bypass slot
823 lead-in flange

### DETAILED DESCRIPTION

Embodiments of the present disclosure will now be described in detail below. Examples of the embodiments are illustrated in the drawings, where the same or similar reference numerals indicate the same or similar parts or parts having the same or similar functions. The embodiments described below with reference to the drawings are merely exemplary; they are intended to explain the present disclosure, and are not to be construed as limiting the present disclosure.

In the description of the present disclosure, unless otherwise expressly specified and limited, the term “connected to each other”, “connected” or “mounted” is to be construed in a broad sense, for example, as mountedly connected, detachably connected, mechanically connected or electrically connected, directly connected to each other or indirectly connected to each other via an intermediary, or internally connected or interactional between two elements. For those of ordinary skill in the art, the preceding terms can be construed according to specific situations in the present disclosure.

In the description of the present disclosure, unless otherwise expressly specified and limited, when a first feature is described as “on” or “below” a second feature, the first feature and the second feature may be in direct contact, or be in contact via another feature between the two features instead of being in direct contact. Moreover, when the first feature is described as “on”, “above” or “over” the second feature, the first feature is right on, above or over the second feature or the first feature is obliquely on, above or over the second feature, or the first feature is simply at a higher level than the second feature. When the first feature is described as “under”, “below” or “underneath” the second feature, the first feature is right under, below or underneath the second feature or the first feature is obliquely under, below or underneath the second feature, or the first feature is simply at a lower level than the second feature.

The technical solutions of the present disclosure are further described below through specific embodiments in conjunction with the drawings.

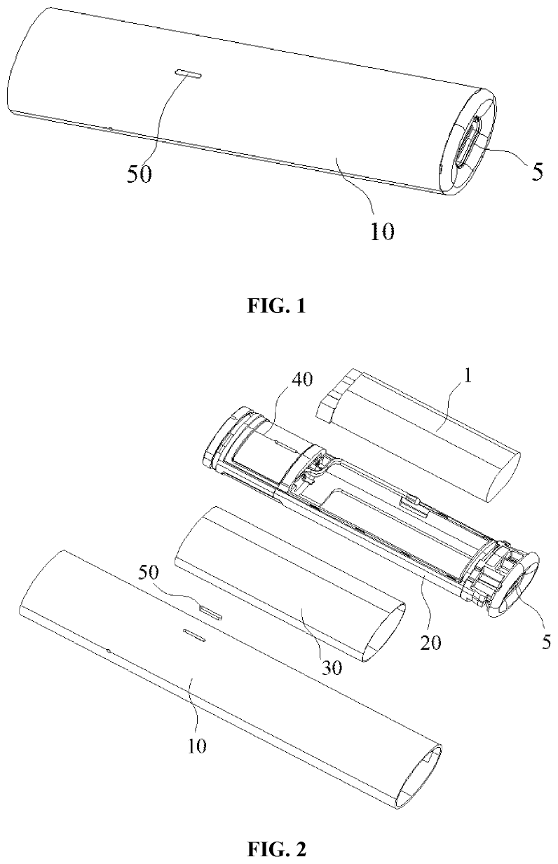

An embodiment of the present disclosure provides a battery module. As shown in FIGS. 1 to 3, the battery module provided in this embodiment includes a main control board 3, a battery 1, and a holder 20. The main control board 3 is electrically connected to the battery 1. The holder 20 is provided with a battery accommodation slot 201 and a main control board accommodation slot 202. The battery accommodation slot 201 and the main control board accommodation slot 202 are sequentially distributed along the length direction of the holder 20. The battery 1 is disposed in the battery accommodation slot 201. The main control board 3 is disposed in the main control board accommodation slot 202.

In an embodiment of the present disclosure, as shown in FIGS. 1 to 3, the battery module provided in this embodiment also includes a battery cover member 30 and a main control board cover plate 40. The battery cover member 30 and the main control board cover plate 40 are separately detachably connected to the holder 20. The battery cover member 30 and the main control board cover plate 40 cover the notch of the battery accommodation slot 201 and the notch of the main control board accommodation slot 202 respectively to fix the positions of the battery 1 and the main control board 3. In this embodiment, the notch of the battery accommodation slot 201 and the notch of the main control board accommodation slot 202 are located on the same side surface of the holder 20.

In an embodiment of the present disclosure, the slot bottom of the battery accommodation slot 201 is provided with an opening 204. The back of the battery 1 can be pressed against through the opening 204 so that the battery 1 can be smoothly taken out from the notch of the battery accommodation slot 201. Therefore, the opening 204 is provided for the convenient detachment of the battery 1.

In an embodiment of the present disclosure, to cover the opening 204 and the notch of the battery accommodation slot 201 simultaneously, the battery cover member 30 in this embodiment is configured to be a sleeve-like structure, and the battery cover member 30 is sleeved outside the battery accommodation slot 201 of the holder 20.

In an embodiment of the present disclosure, the main control board cover plate 40 is detachably connected to the holder 20 by engaging or inserting to facilitate detachment and mounting of the main control board 3, thereby facilitating maintenance or replacement. In this embodiment, insertion protrusions are disposed on the main control board cover plate 40. Insertion slots 203 are disposed in the main control board accommodation slot 202. The insertion protrusions are inserted into the insertion slots 203 to implement the connection between the main control board cover plate 40 and the holder 20.

In an embodiment of the present disclosure, as shown in FIG. 2, the battery module provided in this embodiment also includes a housing 10. The housing 10 is a sleeve structure. The housing 10 is sleeved outside the holder 20, the battery cover member 30, and the main control board cover plate 40.

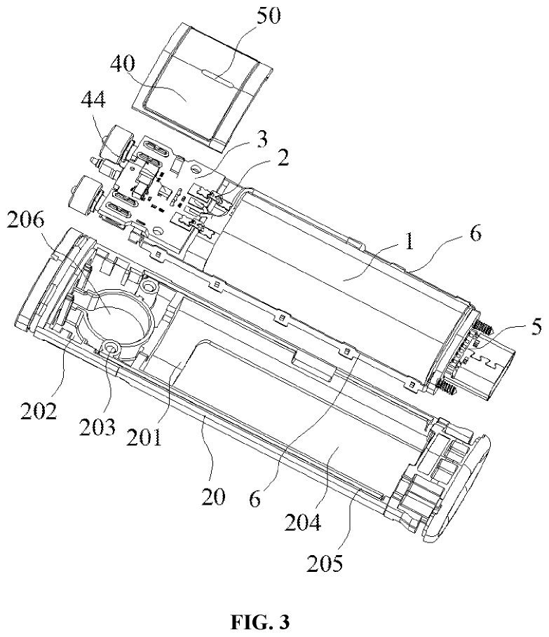

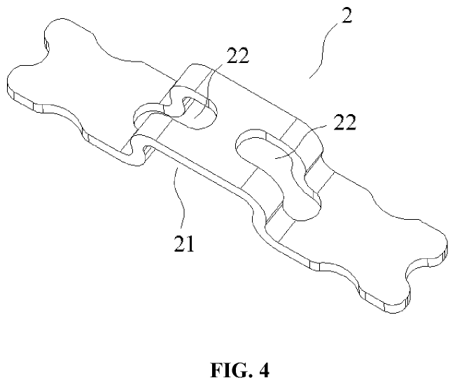

In an embodiment of the present disclosure, as shown in FIG. 3 and FIG. 4, the battery module provided in this embodiment also includes an elastic conduction sheet 2. The battery 1 is electrically connected to the main control board 3 by the elastic conduction sheet 2. One end of the elastic conduction sheet 2 is welded to the battery 1 and another end of the elastic conduction sheet 2 is welded to the main control board 3.

In the battery module provided in this embodiment, the battery 1 is electrically connected to the main control board 3 by the elastic conduction sheet 2 so that conduction between the battery 1 and a circuit of the main control board 3 is achieved. One end of the elastic conduction sheet 2 is welded to the battery 1 and another end of the elastic conduction sheet 2 is welded to the main control board 3 so that two ends of the elastic conduction sheet 2 are connected by welding, thereby eliminating the use of tin and effectively reducing the material costs. Moreover, when the battery 1, the main control board 3, and the elastic conduction sheet 2 have position errors or thermal expansion, the elastic conduction sheet 2 can be elastically deformed adaptively, thereby ensuring the connection firmness of contact points. Additionally, since the ends of the elastic conduction sheet 2 are convenient to position, the welding with the battery 1 and the main control board 3 may be performed through automation equipment, thereby improving the productive efficiency. Therefore, the battery module proposed in this embodiment can reduce the material costs and can be manufactured using automation equipment, thereby increasing the productive efficiency.

In an embodiment of the present disclosure two elastic conduction sheets 2 are provided. Two ends of one elastic conduction sheet 2 are welded to the positive electrode of the battery 1 and the positive electrode of the main control board 3 respectively. Two ends of the other elastic conduction sheet 2 are welded to the negative electrode of the battery 1 and the negative electrode of the main control board 3 respectively.

In an embodiment of the present disclosure, as shown in FIG. 4, the middle portion of an elastic conduction sheet 2 is concave and deformed toward one side to form an elastic bypass slot 21. Two ends of the elastic conduction sheet 2 can be elastically deformed along the direction toward the elastic bypass slot 21 or the direction away from the elastic bypass slot 21. In this manner, the elastic conduction sheet 2 has elasticity.

In an embodiment of the present disclosure, the elastic bypass slot 21 is provided with a through slot 22. The arrangement of the through slot 22 reduces the stiffness at the elastic bypass slot 21 of the elastic conduction sheet 2 and increases the elasticity of the elastic conduction sheet 2. As shown in FIG. 4, two through slots 22 are disposed at the elastic bypass slot 21.

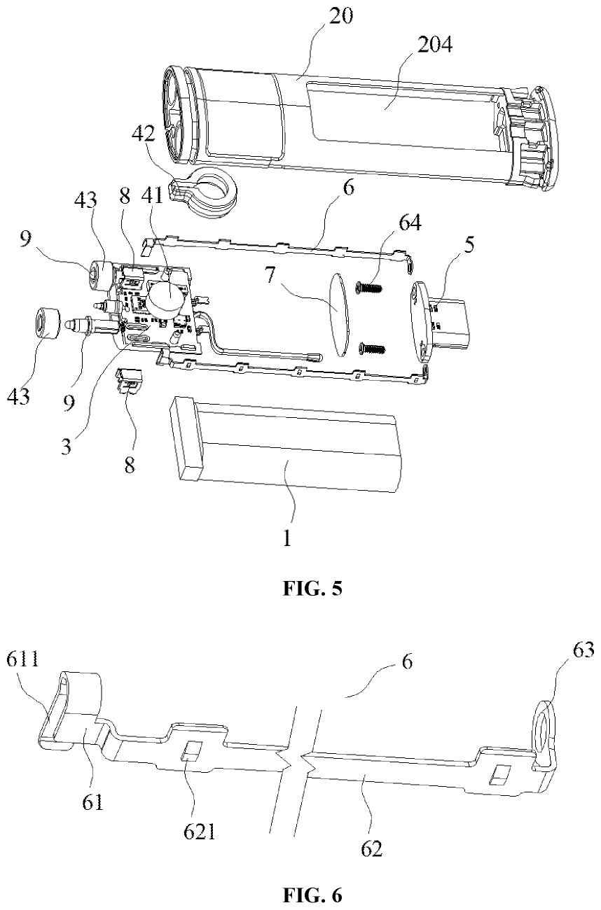

In an embodiment of the present disclosure, as shown in FIG. 5, the battery module provided in this embodiment also includes a charging connector 5 and an electrical connector 6. The charging connector 5 and the main control board 3 are disposed at two ends of the battery 1. The charging connector 5 is electrically connected to the main control board 3 by the electrical connector 6. One end of an electrical connector 6 elastically abuts the main control board 3 and another end of the electrical connector 6 is connected to the charging connector 5. Since one end of the electrical connector 6 elastically abuts the main control board 3, contact loosening due to position errors and thermal expansion of materials is avoided. The tail of the charging connector 5 faces the battery 1 and the head of the charging connector 5 is provided with a charging port. The charging port is exposed outside the holder 20 to charge the battery 1.

In an embodiment of the present disclosure, two electrical connectors 6 are provided. Two electrical connector accommodation slots 205 are disposed on two sides of the battery accommodation slot 201 on the holder 20. Two electrical connectors 6 are disposed in the two electrical connector accommodation slots 205 in a one-to-one manner.

In an embodiment of the present disclosure, as shown in FIG. 5 and FIG. 6, an electrical connector 6 includes a body 62 and an elastic abutment member 61. One end of the body 62 is connected to the charging connector 5. One end of the elastic abutment member 61 is connected to another end of the body 62 and another end of the elastic abutment member 61 is bent and deformed to form an elastic abutment end 611. The elastic abutment end 611 elastically abuts the main control board 3. In an embodiment of the present disclosure, the elastic abutment member 61 has a U-shaped structure. The end of the elastic abutment end 611 is inserted into the main control board 3 and is welded to the main control board 3 so that the connection firmness is ensured, and the costs are reduced because no tin is required.

In an embodiment of the present disclosure, multiple protrusions 621 are disposed on the sidewall of the body 62. The inner wall of an electrical connector accommodation slot 205 of the holder 20 is provided with multiple locking slots. The multiple protrusions 621 engage with the multiple locking slots in a one-to-one manner. In this manner, the firmness of the fixing of the electrical connector 6 is ensured.

In an embodiment of the present disclosure, the electrical connector 6 also includes a fixed portion 63. The fixed portion 63 is connected to one end of the body 62 facing away from the elastic abutment member 61. The body 62 is connected to the charging connector 5 by the fixed portion 63, and the fixed portion 63 is fastened to the charging connector 5 by a fastener 64 such as bolt or screw.

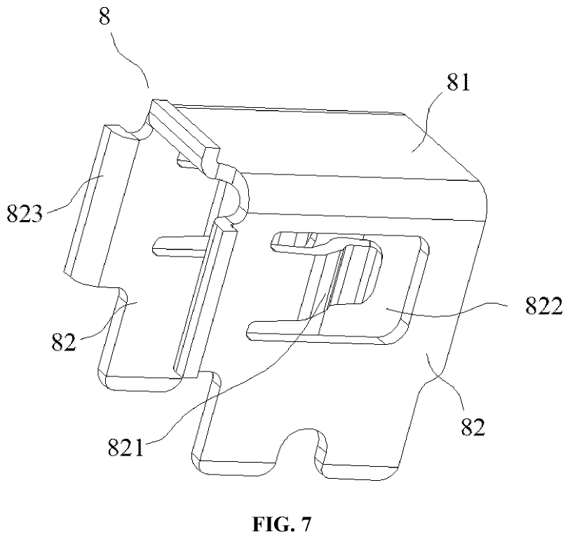

In an embodiment of the present disclosure, as shown in FIG. 5 and FIG. 7, the battery module provided in this embodiment also includes an elastic fastening member 8 and a spring contact terminal 9. The elastic fastening member 8 is connected to the main control board 3. The tail of the spring contact terminal 9 is elastically clamped in the elastic fastening member 8 and the head of the spring contact terminal 9 is configured to contact a power-to-be-supplied component, thereby supplying power to the power-to-be-supplied component. The power-to-be-supplied component may be an atomization compartment or another execution component that requires power. Since the tail of the spring contact terminal 9 is elastically clamped in the elastic fastening member 8, contact loosening due to position errors and thermal expansion of materials is avoided.

In an embodiment of the present disclosure, the elastic fastening member 8 includes a baseplate 81 and two side plates 82. The baseplate 81 and the two side plates 82 are connected to form a U-shaped structure and the two side plates 82 are connected to the main control board 3 and are each provided with an elastic clamping plate 821. The tail of the spring contact terminal 9 is inserted in the U-shaped slot of the U-shaped structure and clamped by two elastic clamping plates 821 of the two side plates 82. Since the baseplate 81 and the two side plates 82 are connected to form a U-shaped structure and the two elastic clamping plates 821 elastically clamps the spring contact terminal, the position of the tail of the spring contact terminal 9 is accordingly limited.

In an embodiment of the present disclosure, the ends of the side plates 82 are inserted into the main control board 3 and welded to the main control board 3 so that the connection firmness is ensured, the tin is eliminated, and the costs are saved.

In an embodiment of the present disclosure, one end of an elastic clamping plate 821 is connected to the inner wall of a side plate 82 and another end of the elastic clamping plate 821 is a free end. The free end extends obliquely toward the inside of the elastic fastening member 8. When the tail of the spring contact terminal 9 is inserted into the elastic fastening member 8 and abuts the free ends of two elastic clamping plates 821, the free ends of the two elastic clamping plates 821 face away from each other and are elastically deformed. The two elastic clamping plates 821 clamp the tail of the spring contact terminal 9 under the action of the elastic restoring force.

In an embodiment of the present disclosure, a side plate 82 is provided with a bypass slot 822. One end of an elastic clamping plate 821 is connected to the inner wall of the bypass slot 822 and another end of the elastic clamping plate 821 is a free end. The free end extends obliquely toward the inside of the elastic fastening member 8, extends out of the bypass slot 822, and enters the U-shaped slot of the elastic fastening member 8 to abut the tail of the spring contact terminal 9. The tail of the spring contact terminal 9 is clamped by two elastic clamping plates 821 of the two side plates 82. In this structure, when manufactured, a part of the side plate 82 can be cut, and the part is bent inwardly to form an elastic clamping plate 821. The exposed through slot is a bypass slot 822. This structure is convenient to manufacture and saves materials. The existence of the bypass slot 822 makes large the elastic deformation range of the elastic clamping plate 821.

In an embodiment of the present disclosure, the ends of the side plates 82 and the baseplate 81 facing the head of the spring contact terminal 9 are bent toward the side facing away from each other to form lead-in flanges 823. The lead-in flanges 823 play a guiding role to facilitate the insertion of the tail of the spring contact terminal 9.

In an embodiment of the present disclosure, as shown in FIG. 5, the battery module provided in this embodiment also includes a shockproof pad 7. The shockproof pad 7 is disposed between the charging connector 5 and the battery 1. The shockproof pad 7 can reduce the vibration of the battery 1 and prevent the abnormal noise from being caused by the vibration.

In an embodiment of the present disclosure, the elastic conduction sheet 2 is made of iron. With respect to the copper wire, the area of the cross section of the elastic conduction sheet 2 is increased, so the electrical conductivity of the elastic conduction sheet 2 is enhanced. Therefore, in the case where the elastic conduction sheet 2 is made of iron, the electrical conductivity is not affected, and the costs are greatly reduced. In this embodiment, the elastic conduction sheet 2 is made of tinplate.

In an embodiment of the present disclosure, as shown in FIG. 5, the main control board 3 is provided with an integrated element 41. The integrated element 41 is an airflow induction and electrical energy control integrated unit and is configured to induct airflow and control circuits. As shown in FIG. 3, the slot bottom of the main control board accommodation slot 202 protrudes to form a partition. The partition encloses an integrated element accommodation slot 206. The integrated element 41 is disposed in the integrated element accommodation slot 206. The main control board 3 is located at the top end of the partition. To ensure the stability of the mounting of the integrated element 41, a soft rubber sealing pad 42 is sealingly disposed between the inner wall of the integrated element accommodation slot 206 and the integrated element 41.

In an embodiment of the present disclosure, as shown in FIG. 5, first magnets 43 are disposed between the holder 20 and spring contact terminals 9. Correspondingly, iron blocks or second magnets that cooperate with the first magnets 43 to adsorb are disposed on a power-to-be-supplied component. When the power-to-be-supplied component is docked with a power supply module, the first magnets 43 adsorb the iron blocks or the second magnets to ensure the stability of the assembly and improve the convenience of assembly.

In an embodiment of the present disclosure, as shown in FIG. 3, an indicator light 44 is disposed on the main control board 3 and a light-transmissive member 50 is embedded in the main control board cover plate 40. The indicator light 44 is opposite to the light-transmissive member 50 and the light of the indicator light 44 is emitted outwardly through the light-transmissive member 50 for a user to understand the working state of a charging module. For example, when the battery 1 is out of power, red light is emitted to remind the battery 1 to charge in time. Green light is emitted to indicate that the battery 1 is full of power. Blue light is displayed when the battery 1 is in a power-consuming state. A specific case may be set according to actual requirements. This is not limited herein.

An embodiment of the present disclosure also provides an atomizer including an atomization compartment and the preceding battery module. The battery module is connected to the atomization compartment and configured to supply power to the atomization compartment. The atomizer may be an air humidifier, a nebulizer, an electronic cigarette, or an atomization device for beauty and cosmetics. The atomizer proposed in the present disclosure includes the preceding battery module. Therefore, the atomizer reduces the material costs and can be manufactured using automation equipment, thereby increasing the productive efficiency.

In an embodiment of the present disclosure, as shown in FIG. 2, the length of a housing 10 is greater than the length of a holder 20. The end of the housing 10 facing away from a charging connector 5 extends beyond the set distance of the end of the holder 20. The extended part forms a mounting slot with the end of the holder 20. One end of the atomization compartment is inserted into the mounting slot to implement assembly of the atomization compartment and the battery module. In this case, the end of the atomization compartment and heads of spring contact terminals 9 are in contact so as to be electrically connected.

What is claimed is:

1. A battery module, comprising a battery, a main control board, and an elastic conduction sheet with elasticity, wherein the battery is electrically connected to the main control board by the elastic conduction sheet, one end of the elastic conduction sheet is welded to the battery, and another end of the elastic conduction sheet is welded to the main control board.

2. The battery module according to claim 1, wherein a middle portion of the elastic conduction sheet is concave and deformed toward one side to form an elastic bypass slot, and two ends of the elastic conduction sheet are capable of being elastically deformed along a direction toward the elastic bypass slot or a direction away from the elastic bypass slot.

3. The battery module according to claim 2, wherein the elastic bypass slot is provided with a through slot.

4. The battery module according to claim 1, further comprising an elastic fastening member and a spring contact terminal, wherein the elastic fastening member is connected to the main control board, a tail of the spring contact terminal is elastically clamped in the elastic fastening member, and a head of the spring contact terminal is configured to contact a power-to-be-supplied component.

5. The battery module according to claim 4, wherein the elastic fastening member comprises a baseplate and two side plates, wherein the baseplate and the two side plates are connected to form a U-shaped structure, the two side plates are connected to the main control board and each provided with an elastic clamping plate, and the tail of the spring contact terminal is inserted in a U-shaped slot of the U-shaped structure and clamped by two elastic clamping plates of the two side plates.

6. The battery module according to claim 1, further comprising a charging connector and an electrical connector, wherein the charging connector and the main control board are disposed at two ends of the battery respectively, the charging connector is electrically connected to the main control board by the electrical connector, one end of the electrical connector elastically abuts the main control board, and another end of the electrical connector is connected to the charging connector.

7. The battery module according to claim 6, wherein the electrical connector comprises a body and an elastic abutment member, wherein one end of the body is connected to the charging connector, one end of the elastic abutment member is connected to another end of the body, another end of the elastic abutment member is bent and deformed to form an elastic abutment end, and the elastic abutment end elastically abuts the main control board.

8. The battery module according to claim 6, further comprising a shockproof pad, wherein the shockproof pad is disposed between the charging connector and the battery.

9. The battery module according to claim 1, wherein the elastic conduction sheet is made of iron.

10. The battery module according to claim 2, wherein the elastic conduction sheet is made of iron.

11. The battery module according to claim 3, wherein the elastic conduction sheet is made of iron.

12. An atomizer, comprising an atomization compartment and a battery module, wherein,
the battery module is connected to the atomization compartment and configured to supply power to the atomization compartment; and
the battery module comprises a battery, a main control board, and an elastic conduction sheet with elasticity, the battery is electrically connected to the main control board by the elastic conduction sheet, one end of the elastic conduction sheet is welded to the battery, and another end of the elastic conduction sheet is welded to the main control board.

13. The atomizer according to claim 12, wherein a middle portion of the elastic conduction sheet is concave and deformed toward one side to form an elastic bypass slot, and two ends of the elastic conduction sheet are capable of being elastically deformed along a direction toward the elastic bypass slot or a direction away from the elastic bypass slot.

14. The atomizer according to claim 13, wherein the elastic bypass slot is provided with a through slot.

15. The atomizer according to claim 12, further comprising an elastic fastening member and a spring contact terminal, wherein the elastic fastening member is connected to the main control board, a tail of the spring contact terminal is elastically clamped in the elastic fastening member, and a head of the spring contact terminal is configured to contact a power-to-be-supplied component.

16. The atomizer according to claim 15, wherein the elastic fastening member comprises a baseplate and two side plates, wherein the baseplate and the two side plates are connected to form a U-shaped structure, the two side plates are connected to the main control board and each provided with an elastic clamping plate, and the tail of the spring contact terminal is inserted in a U-shaped slot of the U-shaped structure and clamped by two elastic clamping plates of the two side plates.

17. The atomizer according to claim 12, further comprising a charging connector and an electrical connector, wherein the charging connector and the main control board are disposed at two ends of the battery respectively, the charging connector is electrically connected to the main control board by the electrical connector, one end of the electrical connector elastically abuts the main control board, and another end of the electrical connector is connected to the charging connector.

18. The atomizer according to claim 17, wherein the electrical connector comprises a body and an elastic abutment member, wherein one end of the body is connected to the charging connector, one end of the elastic abutment member is connected to another end of the body, another end of the elastic abutment member is bent and deformed to form an elastic abutment end, and the elastic abutment end elastically abuts the main control board.

19. The atomizer according to claim 17, further comprising a shockproof pad, wherein the shockproof pad is disposed between the charging connector and the battery.

20. The atomizer according to claim 12, wherein the elastic conduction sheet is made of iron.
